(19) United States
(12) Patent Application Publication
(10) Pub. No.: US 2009/0022826 A1
(43) Pub. Date: Jan. 22, 2009

(54) Multi-step method of pain and/or inflammation treatment

(76) Inventors: David L. Shrier, Brooklyn, NY (US); Joseph P. Grace, Weston, CT (US)

Correspondence Address: KENYON & KENYON LLP, ONE BROADWAY, NEW YORK, NY, 10004, US

(21) Appl. No.: 11/880,287

(22) Filed: Jul. 20, 2007

(51) Int. Cl. A61K 31/16; A61K 31/19; A61K 31/20; A61K 31/355; A61K 36/00; A61K 36/48; A61K 36/55; A61K 36/68; A61K 36/8962; A61K 38/00; A61P 17/00

(52) U.S. Cl. 424/754; 424/725; 424/757; 424/768; 514/12; 514/458; 514/558; 514/568; 514/579; 514/738

(57) ABSTRACT

The present invention provides multi-step methods for treating pain and/or inflammation.

In a preferred embodiment, the method comprises administering to a patient in need of relief of pain and/or inflammation the following three components:
(1) a skin penetration enhancer;
(2) a daytime analgesic; and
(3) a nighttime joint and muscle rejuvenator.

The present invention also provides an analgesic composition comprising PHYTOLANE LS® and garlic oil, preferably in a cream or ointment base.

### FIELD OF THE INVENTION

The present invention relates to topical methods for treating pain and/or inflammation.

### BACKGROUND OF THE INVENTION

U.S. Pat. No. 5,032,400 describes a composition for the topical treatment of pain and/or inflammation comprising shark liver oil and garlic oil in a cream or ointment base.

### SUMMARY OF THE INVENTION

The present invention provides multi-step methods for treating pain and/or inflammation comprising the topical administration of certain components.

In a preferred embodiment, the method comprises administering to a patient in need of relief of pain and/or inflammation the following components, in the following order:

(1) a skin penetration enhancer;

(2) a daytime analgesic;

(3) the skin penetration enhancer; and

(4) a nighttime joint and muscle rejuvenator.

The present invention provides safe and effective relief of pain and/or inflammation caused by such conditions as arthritis, bursitis, or neuritis as well as pain and/or inflammation due to strains and sprains from such causes as sports injuries or other injuries.

### DETAILED DESCRIPTION OF THE INVENTION

As used herein the term “pharmaceutically acceptable” means not biologically or otherwise undesirable, i.e., can be administered to an individual without causing significant undesirable effects.

As used herein the term “analgesic” means a component of the pain treatment methods described herein that, when applied to the skin of a patient, reduces pain and/or inflammation.

The present invention is directed to the use of a skin penetration enhancer in combination with a topical analgesic to treat pain and/or inflammation. The present invention also is directed to the combination of a day-cream and a night-cream with different effects (a “day and night” system for 24-hour pain relief).

In one embodiment, the present invention provides a multi-step method of treating pain and/or inflammation comprising administering the following three components to a patient in need of relief from pain and/or inflammation:

(1) a skin penetration enhancer;

(2) a daytime analgesic; and

(3) a nighttime joint and muscle rejuvenator.

The present multi-step method has the following features:

(a) a three component system presented as a “pain regimen” rather than a solo pain product;

(b) separate day and night components for 24 relief;

(c) a penetration enhancer (component 1) to enhance penetration/delivery of components 2 and 3;

(d) a combination of OTC medicine and skincare ingredients to simultaneously treat pain while nurturing skin around troubled areas.

The components of the multi-step method are described in more detail below.

(1) Skin Penetration Enhancer

The skin penetration enhancer can be in the form of a liquid that is dispensed from a tube or bottle via a pump. As dispensed, the skin penetration enhancer can be a light foam. The skin penetration enhancer opens pores and pathways in the skin, thereby improving the delivery of the daytime analgesic and the nighttime joint and muscle rejuvenator.

In one embodiment, a suitable formulation for the skin penetration enhancer can include some or all of the following ingredients: Water, Ethoxydiglycol, Sodium Cocoyl Glutamate, Disodium EDTA, Citric Acid, Phenoxyethanol, Caprylyl Glycol, Ethylhexylglycerin, Hexylene Glycol.

Especially preferred are skin penetration enhancers that contain Ethoxydiglycol. Ethoxydiglycol is available commercially under the brand name TRANSCUTOL®. TRANSCUTOL® is purified diethylene glycol monoethyl ether

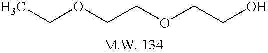

Ethoxydiglycol is a clear liquid that is soluble in both water and alcohol and partially soluble in vegetable oil. It is insoluble in mineral oil. It is believed to increase skin penetration by creating new hydrophilic pathways in the skin and to increase the solubility of medicinal substances by disorganizing the lipid chains of the medicinal substances, thereby increasing the substances' mobility.

A pharmaceutical formulator will be able to choose from among the ingredients listed above, and others known in the art, the proper blend and appropriate amounts of these or similar ingredients to produce a suitable skin penetration enhancer, keeping in mind the function of the skin penetration enhancer, i.e., to enhance penetration/delivery of the daytime analgesic and the nighttime joint and muscle rejuvenator.

Skin penetration enhancers that are known in the art are also suitable for use with the present methods.

(2) Daytime Analgesic

The daytime analgesic is a topical pain remedy. It is intended to be administered during the daytime, i.e., during the patient's waking hours, well before the patient goes to bed for the night. It contains two main ingredients—Bryonia and Rhus toxicodendron. Preferably, the Bryonia is Bryonia 6×, about 0.2% (v/v) and the Rhus toxicodendron is Rhus toxicodendron 6×, about 0.2% (v/v). Preparations of Bryonia and Rhus toxicodendron such as Bryonia 6× and Rhus toxicodendron 6× are well known in the homeopathic art and methods for their preparation are widely known. Furthermore, they are available from a large number of commercial suppliers. In general, Bryonia 6× is prepared by taking a drop of Bryonia extract, prepared according to methods known in the homeopathic art, and diluting it in 100 gallons of water. This is repeated five times to give Bryonia 6×. Rhus toxicodendron 6× is prepared in a similar manner.

The remaining ingredients in the daytime analgesic can include all or some of the following: Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, Fragrance.

Preferably, the daytime analgesic includes squalane, preferably in an amount of about 2% (v/v). Preferably, the squalane does not have a fishy odor. One such suitable form of squalane is available commercially as PHYTOLANE LS® from Barnet Products Corporation of Englewood Cliffs, N.J. PHYTOLANE LS® (CAS: 111-01-3) is a particular saturated form of squalene having the structure

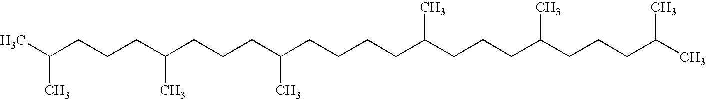

PHYTOLANE LS® is a natural emollient that readily forms an emulsion with fixed oils and lipophilic substances. It is colorless, tasteless, and transparent.

In certain embodiments, the remaining ingredients of the daytime analgesic include flax seed oil (i.e., linseed oil), preferably at about 5% (v/v) of the final daytime analgesic. In such embodiments containing flax seed oil, the remaining ingredients in the daytime analgesic can include all or some of the following: Water, Emulsifying Wax, Linum Usitatissimum (Linseed) Seed Oil, Propylene Glycol, Squalane, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, Fragrance.

A pharmaceutical formulator will be able to choose from among the above-listed remaining ingredients, and others known in the art, the proper blend and appropriate amounts to produce a suitable daytime analgesic. Alternatively, the daytime analgesic may be a topical analgesic preparation containing acetylsalicylic acid (aspirin) or another topical analgesic preparation.

In certain embodiments, the daytime analgesic is the topical analgesic described in U.S. Pat. No. 5,032,400.

In related embodiments, the daytime analgesic is a novel analgesic for topical application containing a combination of PHYTOLANE LS® and garlic oil in a cream or ointment base. In certain embodiments, the novel daytime analgesic comprises at least the following three or more ingredients together in therapeutic quantities: garlic oil, PHYTOLANE LS®, and soybean oil. These ingredients can be formulated together with emulsifiers, fragrances, and other ingredients into an ointment or a cream for topical application for the treatment of pain and/or inflammation.

Garlic oil is well-known in the art and can be prepared from the fresh bulbs of Allium sativum as a yellowish, volatile oil containing various sulfur compounds. Any unpleasant odor can be masked with a suitable fragrance or fragrances.

The preferred form of this daytime analgesic is an emulsion of an oil phase dispersed in an aqueous phase. The oil phase may include emulsifying agents, e.g., emulsifying wax N.F., a waxy solid prepared from cetostearyl alcohol containing a polyoxyethylene derivative of a fatty acid and its salts, a mixture of stearic and palmitic acids, that is commonly used as a base in ointments, creams and cosmetics.

Myristic acid, its esters and alcohols, as well as other fat sources used as emollients in cream bases may be included. Alpha tocopherol (Vitamin E) or other substances used as antioxidants for plant and animal oils, protectives, stabilizers, preservatives, and the like may also be present.

The aqueous phase of the emulsion contains primarily water, such as distilled or deionized water. The aqueous phase may also contain a compatible, water-soluble humectant, such as propylene glycol, together with emulsifiers and dispersants.

In certain embodiments, the present invention provides an analgesic composition comprising, by weight, 2-12% PHYTOLANE LS® and 0.05-0.5% garlic oil in a cream or ointment base. In certain embodiments, the analgesic composition also comprises soybean oil at 2-10% by weight and/or almond oil at 2-8% by weight.

In certain embodiments, the present invention provides an analgesic composition comprising, in percent by weight:

| | |
|---|---|
| an emulsifying agent | 1-8 |
| garlic oil | 0.05-0.5 |
| stearic acid | 1-3 |
| PHYTOLANE LS ® | 2-12 |
| almond oil | 2-8 |
| myristylmyristate | 0.5-2 |
| soybean oil | 2-10 |
| propyl paraben | 0.05-0.5 |
| alpha tocopherol | 1-6 |
| water | 40-70 |
| an alkali soluble acrylic | 0.1-0.8 |
| polymer emulsion | |
| propylene glycol | 2-8 |
| methyl paraben | 0.05-0.6 |
| diazolidinyl urea and parabens | 0.05-0.6 |
| triethanol amine 99% | 0.5-2.5. |

In certain embodiments, the analgesic composition also comprises, in percent by weight:

| | |
|---|---|
| soluble reticulin | 0.05-2 |
| hydrolyzed elastin | 0.5-2 |
| fragrance | 0.1-6 |

In certain embodiments, the emulsifying agent is an emulsifying wax such as N.F. XVIII.

In certain embodiments, the an alkali soluble acrylic polymer emulsion is Acrysol ICS-1.

In certain embodiments, the fragrance is Alpine fragrance 161-742.

The present invention provides methods of preparing analgesic compositions comprising PHYTOLANE LS® and garlic oil. In a particular embodiment, the method comprises combining PHYTOLANE LS® and garlic oil in a cream or ointment base.

In another embodiment, the present invention provides a method of preparing an analgesic composition comprising:
(a) combining PHYTOLANE LS® and garlic oil to form a first mixture;
(b) combining water and an alkali soluble acrylic polymer emulsion to form a second mixture; and
(c) combining the first mixture and the second mixture to form the analgesic composition.

Optionally, soybean oil and/or an emulsifying agent are also combined with the PHYTOLANE LS® and the garlic oil in step (a).

The present invention also provides a method of preparing an analgesic composition comprising:
(a) combining PHYTOLANE LS® and garlic oil to form a first mixture;
(b) combining water and an alkali soluble acrylic polymer emulsion to form a second mixture;
(c) combining soluble reticulin, hydrolyzed elastin, and a fragrance to form a third mixture;
(d) combining the first mixture and the second mixture to form a fourth mixture; and
(e) combining the third mixture and the fourth mixture to form the analgesic composition.

The present invention provides a method of treating pain or inflammation in a patient in need thereof comprising applying an analgesic composition comprising PHYTOLANE LS® and garlic oil in a cream or ointment base to a selected area of skin of the patient. In certain embodiments, the analgesic composition also comprises soybean oil and/or almond oil. In certain embodiments, the analgesic composition is applied to the selected area of skin one to four times per day.

The final form of the daytime analgesic is preferably that of an ointment or a cream. The ointment or cream may be an emulsion having an oil phase dispersed with suitable emulsifiers and stabilizers into an aqueous phase.

The desired volume of the daytime analgesic may be adjusted by using appropriate amounts of neutral oils, e.g., soybean oil. The oils and other excipients are preferably compatible with each other, pharmaceutically acceptable, and preferably are safe enough to be edible. It is also preferred that they be readily available and inexpensive.

The daytime analgesic functions to provide pain and/or anti-inflammatory relief to the portion of the body to which it is applied.

(3) Nighttime Joint and Muscle Rejuvenator

The nighttime joint and muscle rejuvenator is intended to be administered in the evening, i.e., fairly soon before the patient goes to bed for the night. The ingredients in the nighttime joint and muscle rejuvenator can include all or some of the following: Water, Caprylic/Capric/Stearic Triglyceride, Cetearyl Alcohol, Neopentyl Glycol Diheptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, Hexylene Glycol.

In certain embodiments, the nighttime joint and muscle rejuvenator contains Bryonia 6×, about 0.2% (v/v) and Rhus toxicodendron 6×, about 0.2% (v/v).

A pharmaceutical formulator will be able to choose from among these ingredients, and others known in the art, the proper blend and appropriate amounts to produce a suitable nighttime joint and muscle rejuvenator. The final form of the nighttime joint and muscle rejuvenator is preferably that of an ointment or a cream.

The nighttime joint and muscle rejuvenator may provide additional pain and inflammation relief. The nighttime joint and muscle rejuvenator may also function as a skin moisturizer and a counterirritant. It may soothe the skin and counteract any irritation that might be caused by the daytime analgesic.

Preferably, the nighttime joint and muscle rejuvenator contains different ingredients from those of the daytime analgesic and provides a different effect from that of the daytime analgesic.

The components of the methods of the present invention are applied to the skin in the area of discomfort, pain, and/or inflammation. The methods begin with rubbing the skin penetration enhancer onto the area of skin where treatment is desired. The skin penetration enhancer is generally dispensed via a pump mechanism from a tube or bottle. One pump per application of the skin penetration enhancer is usually sufficient. This represents about 0.3 to about 1.2, preferably about 0.4 to about 1.0, more preferably about 0.6 to about 0.8, and most preferably about 0.7 ounces of skin penetration enhancer by weight per application. Expressed in metric units, about 8.5 to about 34, preferably about 11.4 to about 28.5, more preferably about 17 to about 23, and most preferably about 20 grams of skin penetration enhancer can be used per application. Of course, somewhat more or somewhat less may also be applied.

Application of the skin penetration enhancer is followed, more or less immediately, by application of the daytime analgesic to the same area of skin. Generally, one application of daytime analgesic uses about 0.3 to about 1.2, preferably about 0.4 to about 1.0, more preferably about 0.6 to about 0.8, and most preferably about 0.7 ounces of daytime analgesic by weight. Expressed in metric units, about 8.5 to about 34, preferably about 11.4 to about 28.5, more preferably about 17 to about 23, and most preferably about 20 grams of daytime analgesic can be used per application. Of course, somewhat more or somewhat less may also be applied. The daytime analgesic can be applied as many times during the day as desired. The combination of skin penetration enhancer and daytime analgesic may be applied as many times as desired, depending upon pain and/or inflammation level.

In some cases, the daytime analgesic may be applied without first applying the skin penetration enhancer.

The next step is to apply the nighttime joint and muscle rejuvenator to the same area of skin to which the skin penetration enhancer and the daytime analgesic were applied. In most cases, the skin penetration enhancer is again applied to the same area of skin. This is followed, more or less immediately, by application of the nighttime joint and muscle rejuvenator to the same area of skin. Generally, one application of nighttime joint and muscle rejuvenator uses about 0.3 to about 1.2, preferably about 0.4 to about 1.0, more preferably about 0.6 to about 0.8, and most preferably about 0.7 ounces of nighttime joint and muscle rejuvenator by weight. Expressed in metric units, about 8.5 to about 34, preferably about 11.4 to about 28.5, more preferably about 17 to about 23, and most preferably about 20 grams of nighttime joint and muscle rejuvenator can be used per application. Of course, somewhat more or somewhat less may also be applied.

In some cases, the nighttime joint and muscle rejuvenator may be applied without first applying the skin penetration enhancer.

Preferably, the nighttime joint and muscle rejuvenator is applied just before, or fairly soon before the patient goes to bed. Preferably, the nighttime joint and muscle rejuvenator is applied a few minutes (e.g., about 1 to 60 minutes, about 1 to 30 minutes, about 1 to 20 minutes, or about 1, 2, 3, 4, 5, 6, 7, 8, 9, or 10 minutes) before going to bed.

The components of the multi-step method may be applied by the patient, or they may be applied to the patient by a care giver.

The methods of the present invention are especially suitable for treating pain, joint stiffness, soreness, and/or inflammation due to injuries to skeletal muscles arising from strains, sprains, rheumatoid, musculoskeletal, and osteoarthritis, sports injuries, etc. The methods also can be used to treat pain, soreness, and/or inflammation in joints, tendons, or nerves.

The multi-step methods of the present invention, when topically applied to the affected area, provide relief of pain, stiffness, soreness and swelling in both shallow and deep body tissues. The multi-step methods may be used for the temporary or long term treatment of inflammatory skeletal muscle conditions such as arthritis, bursitis, neuritis, strains, sprains, and other sports injuries.

In contrast to most prior art pain treatments, the multi-step system of the present invention is a “pain treatment regimen” rather than a solo pain product. In some embodiments, the multi-step methods provide a combination of OTC medicine and skincare ingredients to simultaneously treat pain while nurturing skin around troubled areas.

Patients to which the multi-step methods of the present invention may be applied include mammals, particularly humans, but also dogs, cats, horses, cattle, etc.

The present invention provides a method for treating pain or inflammation in a patient in need thereof comprising, in the following order:

(a) applying a skin penetration enhancer to a selected area of skin of the patient;

(b) applying a daytime analgesic to the selected area of skin;

(c) applying the skin penetration enhancer to the selected area of skin; and

(d) applying a nighttime joint and muscle rejuvenator to the selected area of skin.

In certain embodiments, steps (a) and (b) are repeated once, twice, three times, or more, before step (c) is carried out.

In certain embodiments, the time period between step (b) and step (c) is at least about 4 hours to about 16 hours, preferably about 6 hours to about 14 hours, and more preferably about 8 hours to about 12 hours. In certain embodiments, the time period between step (b) and step (c) is at least about 4 hours, 6 hours, 8 hours, 10 hours, 12 hours, 14 hours, or 16 hours.

In certain embodiments, the time period between steps (a) and (b) as well as the time period between steps (c) and (d) is more or less immediate, or is about 5 seconds to about 5 minutes, preferably about 10 seconds to about 2 minutes, more preferably about 10 seconds to about 1 minute.

In certain embodiments, step (a) or (c), or both, are omitted. Thus, the present invention also includes:

A method for treating pain or inflammation in a patient in need thereof comprising, in the following order:

(a) applying a daytime analgesic to a selected area of skin of the patient;

(b) applying a skin penetration enhancer to the selected area of skin; and

(c) applying a nighttime joint and muscle rejuvenator to the selected area of skin.

A method for treating pain or inflammation in a patient in need thereof comprising, in the following order:

(a) applying a skin penetration enhancer to a selected area of skin of the patient;

(b) applying a daytime analgesic to the selected area of skin; and

(c) applying a nighttime joint and muscle rejuvenator to the selected area of skin.

A method for treating pain or inflammation in a patient in need thereof comprising, in the following order:

(a) applying a daytime analgesic to a selected area of skin of the patient; and

(b) applying a nighttime joint and muscle rejuvenator to the selected area of skin.

In certain embodiments, the steps of the above-described methods are carried out within a 24-hour period, preferably within one waking cycle of the patient.

In certain embodiments, the daytime analgesic and nighttime joint and muscle rejuvenator both contribute to the relief of pain and/or inflammation. In certain embodiments, the daytime analgesic and nighttime joint and muscle rejuvenator contain different ingredients.

In certain embodiments of the above-described methods, the skin penetration enhancer comprises ingredients selected from the group consisting of: Water, Ethoxydiglycol, Sodium Cocoyl Glutamate, Disodium EDTA, Citric Acid, Phenoxyethanol, Caprylyl Glycol, Ethylhexylglycerin, and Hexylene Glycol.

In certain embodiments, the skin penetration enhancer comprises the ingredients Water, Ethoxydiglycol, Sodium Cocoyl Glutamate, Disodium EDTA, Citric Acid, Phenoxyethanol, Caprylyl Glycol, Ethylhexylglycerin, and Hexylene Glycol.

In certain embodiments, the daytime analgesic comprises Bryonia 6× and Rhus toxicodendron 6×. In certain embodiments, the daytime analgesic contains about 0.2% (v/v) each of Bryonia 6× and Rhus toxicodendron 6×.

Bryonia 6× and Rhus toxicodendron 6×, as well as other ingredients referred to herein, are described in such standard texts as The Homeopathic Pharmacopeia of the United States (HPUS) and the Materia Medica. Methods of preparing Bryonia 6× and Rhus toxicodendron 6×, as well as other ingredients referred to herein, are also described in such standard texts as The Homeopathic Pharmacopeia of the United States (HPUS) and the Materia Medica.

In certain embodiments, the daytime analgesic comprises ingredients selected from the group consisting of: Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, and Fragrance.

In certain embodiments, the daytime analgesic comprises the ingredients Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, and Fragrance.

In certain embodiments, the nighttime joint and muscle rejuvenator comprises ingredients selected from the group consisting of: Water, Caprylic/Capric/Stearic Triglyceride, Cetearyl Alcohol, Neopentyl Glycol Diheptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, and Hexylene Glycol.

In certain embodiments, the nighttime joint and muscle rejuvenator comprises the ingredients Water, Caprylic/Capric/Stearic Triglyceride, Cetearyl Alcohol, Neopentyl Glycol Diheptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, and Hexylene Glycol.

In certain embodiments, the present methods are used to treat pain and/or inflammation caused by arthritis, bursitis, neuritis, carpal tunnel syndrome, muscle soreness, repetitive motion injuries, premenstrual cramps, gout, strains, or sprains. In particular, the present methods are suitable for treating rheumatoid arthritis, osteoarthritis, and musculoskeletal arthritis. The present methods may also be used to treat other forms of arthritis or related diseases and conditions, including: Achilles tendinitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendinitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costostemal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis, Hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome. Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy. Nodular panniculitis. Ochronosis. Olecranon bursitis. Osgood-Schlatter's disease, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendinitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid vasculitis, Rotator cuff tendinitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease, Yersinial arthritis.

In certain embodiments of the present methods, the patient is a human.

In certain embodiments, the patient is suffering from pain, joint stiffness, soreness, and/or inflammation due to injuries to skeletal muscles arising from strains, sprains, arthritis, or sports injuries.

The present invention also provides a kit comprising a container or package containing a skin penetration enhancer, a daytime analgesic, and a nighttime joint and muscle rejuvenator. Optionally, the kit or package is divided into at least three compartments or portions and each of the three compartments or portions contains either a skin penetration enhancer, a daytime analgesic, or a nighttime joint and muscle rejuvenator. Preferably, the kit or package also contains a printed set of instnictions describing how to implement the multi-step method.

The present invention also provides a method for treating pain or inflammation in a patient in need thereof comprising:

(a) applying a skin penetration enhancer to a selected area of skin of the patient;

(b) applying a topical analgesic to the selected area of skin.

The composition of the skin penetration enhancer is as described above. The composition of the analgesic is as described above for the daytime analgesic. In preferred embodiments, skin penetration enhancer and the topical analgesic are separate components and contain different ingredients.

Having described the invention with reference to certain preferred embodiments, other embodiments will become apparent to one skilled in the art from consideration of the specification. The invention is further defined by reference to the following example describing the invention in detail. It will be apparent to those skilled in the art that many modifications, both to the materials and methods described herein, may be practiced without departing from the scope of the invention.

The following examples are given for the purpose of illustrating the invention and shall not be construed as limiting the scope or spirit of the invention.

### EXAMPLES

### Example 1

In a preferred embodiment, the present invention provides a three-component system for treating pain and/or inflammation that comprises:

(1) a skin penetration enhancer comprising Water, Ethoxydiglycol, Sodium Cocoyl Glutamate, Disodium EDTA, Citric Acid, Phenoxyethanol, Caprylyl Glycol, Ethylhexylglycerin, Hexylene Glycol;

(2) a daytime analgesic comprising: Bryonia 6×, about 0.2% (v/v) and Rhus toxicodendron 6×, about 0.2% (v/v), Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, Fragrance; and

(3) a nighttime joint and muscle rejuvenator comprising: Water, Caprylic/Capric/Stearic Triglyceride, Cetearyl Alcohol, Neopentyl Glycol Diheptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, Hexylene Glycol.

### Example 2

A. The following ingredients were mixed together in the order listed and in the amounts indicated:

| Ingredient | % by weight |
|---|---|
| emulsifying wax, e.g., N.F. XVIII | 1-8 |
| garlic oil | 0.05-0.5 |
| stearic acid | 1-3 |
| PHYTOLANE LS ® | 2-12 |
| almond oil | 2-8 |
| myristylmyristate | 0.5-2 |
| soybean oil | 2-10 |
| propyl paraben | 0.05-0.5 |
| alpha tocopherol | 1-6 |

The resulting mixture was heated to 80° C.

B. In a separate container, the following ingredients were mixed together in the order listed and in the amounts indicated:

| Ingredient | % by weight |
|---|---|
| distilled water | 40-70 |
| alkali soluble acrylic polymer emulsion, | 0.1-0.8 |
| e.g., Acrysol ICS-1 | |
| propylene glycol | 2-8 |
| methyl paraben | 0.05-0.6 |
| diazolidinyl urea and parabens | 0.05-0.6 |
| triethanol amine 99% | 0.5-2.5 |

The resulting mixture was heated to 90° C.

The heated mixtures from parts A and B were mixed with each other slowly with rapid agitation until mixing was complete and the resulting product was uniform. The product of A and B was then cooled to 40° C.

C. In a separate container the following ingredients are mixed together in the order listed and in the amounts indicated:

| Ingredient | % by weight |
|---|---|
| soluble reticulin | 0.05-2 |
| hydrolyzed elastin | 0.5-2 |
| fragrance, e.g., Alpine fragrance 161-742 | 0.1-6 |

The individual ingredients, 18 in all, total 100%. Soluble reticulin is a collagen compound which forms a network to build up connective tissue. It cooperates with elastin in the major elastic protein of collagen.

The mixture from part C was added to the previously-mixed product of A and B. The mixture of A, B, and C was then mixed until uniform and cooled to room temperature (about 18-23° C.). The final product was a tan colored, creamy oleaginous semi-solid resembling face cream with a slightly peach smell and a pH of between 7.5 and 8.0.

What is claimed is:

1. A method for treating pain or inflammation in a patient in need thereof comprising, in the following order:
(a) applying a skin penetration enhancer to a selected area of skin of the patient;
(b) applying a daytime analgesic to the selected area of skin;
(c) applying the skin penetration enhancer to the selected area of skin; and
(d) applying a nighttime joint and muscle rejuvenator to the selected area of skin.

2. The method of claim 1 where the daytime analgesic comprises Bryonia and Rhus toxicodendron.

3. The method of claim 2 where the daytime analgesic comprises Bryonia 6× and Rhus toxicodendron 6×, each present at about 2% (v/v).

4. The method of claim 3 where the daytime analgesic comprises squalane at about 2% (v/v).

5. The method of claim 3 where:
(i) the skin penetration enhancer comprises ingredients selected from the group consisting of: Water, Ethoxydiglycol, Sodium Cocoyl Glutamate, Disodium EDTA, Citric Acid, Phenoxyethanol, Caprylyl Glycol, Ethylhexylglycerin, and Hexylene Glycol;
(ii) the daytime analgesic comprises ingredients selected from the group consisting of: Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, and Fragrance; and
(iii) the nighttime joint and muscle rejuvenator comprises ingredients selected from the group consisting of: Water, Caprylic/Capric/Stearic Triglyceride, Cetearyl Alcohol, Neopentyl Glycol Dibeptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, and Hexylene Glycol.

6. The method of claim 3 where:
(i) the skin penetration enhancer comprises Water, Ethoxydiglycol, Sodium Cocoyl Glutamate, Disodium EDTA, Citric Acid, Phenoxyethanol, Caprylyl Glycol, Ethylhexylglycerin, and Hexylene Glycol;
(ii) the daytime analgesic comprises Water, Squalane, Emulsifying Wax, Propylene Glycol, Prunus Amygdalus Dulcis (Sweet Almond) Oil, Glycine Soja (Soybean) Oil, Stearic Acid, Tocopherol, Menthol, Myristyl Myristate, Collagen, Hydrolyzed Elastin, Aloe Barbadensis Leaf, Allium Sativum (Garlic) Bulb Extract, Cocos Nucifera (Coconut) Oil, Mineral Oil, Triethanolamine, Diazolidinyl Urea, Methylparaben, Propylparaben, and Fragrance; and
(iii) the nighttime joint and muscle rejuvenator comprises Water, Caprylic/Capric/Stearic Triglyceride, Cetearyl Alcohol, Neopentyl Glycol Diheptanoate, Cetyl Tallowate, Dimethyl Sulfone, Menthol, Potassium Jojobate, Jojoba Alcohol, Glucosamine HCl, Sodium Chondroitin Sulfate, Chondroitin Sulfate, Tetrahexyldecyl Ascorbate, Tocopheryl Acetate, Capsicum Frutescens Fruit Extract, Camellia Sinensis Leaf Extract, Aloe Barbadensis Leaf Juice, Squalane, Zinc PCA, Simethicone, Mentha Piperita (Peppermint) Oil, Camphor, Lecithin, Glycerin, Butylene Glycol, Carbomer, Inulin Lauryl Carbamate, Disodium EDTA, Sodium Hydroxide, Phenoxyethanol, Caprylyl Glycol, Ethylhexylglycerin, and Hexylene Glycol.

7. The method of claim 5 where the nighttime joint and muscle rejuvenator is applied about 8 hours to about 12 hours after the daytime analgesic is applied.

8. The method of claim 6 where the nighttime joint and muscle rejuvenator is applied about 1 to about 60 minutes before going to bed.

9. The method of claim 8 where the patient is a human.

10. The method of claim 9 where the patient is suffering from pain, joint stiffness, soreness, and/or inflammation due to injuries to skeletal muscles arising from strain, sprains, or sports injuries.

11. The method of claim 9 where the patient is suffering from arthritis.

12. The method of claim 9 where the daytime analgesic and the nighttime joint and muscle rejuvenator are creams or ointments.

13. The method of claim 4 where the daytime analgesic contains flax seed oil.

14. A method for treating pain or inflammation in a patient in need thereof comprising, in the following order:
(a) applying a daytime analgesic to a selected area of skin of the patient; and
(b) applying a nighttime joint and muscle rejuvenator to the selected area of skin.

15. A method for treating pain or inflammation in a patient in need thereof comprising:
(a) applying a skin penetration enhancer to a selected area of skin of the patient; and
(b) applying a topical analgesic to the selected area of skin;
where the skin penetration enhancer and the topical analgesic are separate components and contain different ingredients.

16. A kit comprising a container or package divided into at least three compartments or portions where each of the three compartments or portions contains either a skin penetration enhancer, a daytime analgesic, or a nighttime joint and muscle rejuvenator.

17. An analgesic composition comprising PHYTOLANE LS® and garlic oil in a cream or ointment base.

18. The analgesic composition of claim 17 where PHYTOLANE LS® is present at 2-12% by weight and garlic oil is present at 0.05-0.5% by weight.

19. The analgesic composition of claim 18 further comprising soybean oil at 2-10% by weight.

20. The analgesic composition of claim 19 further comprising almond oil at 2-8% by weight.

21. An analgesic composition comprising, in percent by weight:

| | |
|---|---|
| an emulsifying wax | 1-8 |
| garlic oil | 0.05-0.5 |
| stearic acid | 1-3 |
| PHYTOLANE LS ® | 2-12 |
| almond oil | 2-8 |
| myristylmyristate | 0.5-2 |
| soybean oil | 2-10 |
| propyl paraben | 0.05-0.5 |
| alpha tocopherol | 1-6 |
| water | 40-70 |
| an alkali soluble acrylic | 0.1-0.8 |
| polymer emulsion | |
| propylene glycol | 2-8 |
| methyl paraben | 0.05-0.6 |
| diazolidinyl urea and parabens | 0.05-0.6 |
| triethanol amine 99% | 0.5-2.5. |

22. The analgesic composition of claim 21 further comprising, in percent by weight:

| | |
|---|---|
| soluble reticulin | 0.05-2 |
| hydrolyzed elastin | 0.5-2 |
| fragrance | 0.1-6. |

23. The analgesic composition of claim 22 where the emulsifying wax is N.F. XVIII, the an alkali soluble acrylic polymer emulsion is Acrysol ICS-1, and the fragrance is Alpine fragrance 161-742.

24. The analgesic composition of claim 17 which is an emulsion of an oil phase dispersed in an aqueous phase.

25. The analgesic composition of claim 21 which is an emulsion of an oil phase dispersed in an aqueous phase.

26. A method of preparing an analgesic composition comprising combining PHYTOLANE LS® and garlic oil in a cream or ointment base.

27. A method of preparing an analgesic composition comprising:
(a) combining PHYTOLANE LS® and garlic oil to form a first mixture;
(b) combining water and an alkali soluble acrylic polymer emulsion to form a second mixture; and
(c) combining the first mixture and the second mixture to form the analgesic composition.

28. The method of claim 27 where soybean oil is also combined with the PHYTOLANE LS® and the garlic oil in step (a).

29. A method of preparing an analgesic composition comprising:
(a) combining PHYTOLANE LS® and garlic oil to form a first mixture;
(b) combining water and an alkali soluble acrylic polymer emulsion to form a second mixture;
(c) combining soluble reticulin, hydrolyzed elastin, and a fragrance to form a third mixture;
(d) combining the first mixture and the second mixture to form a fourth mixture; and
(e) combining the third mixture and the fourth mixture to form the analgesic composition.

30. A method of treating pain or inflammation in a patient in need thereof comprising applying an analgesic composition comprising PHYTOLANE LS® and garlic oil in a cream or ointment base to a selected area of skin of the patient.

31. The method of claim 30 where the analgesic composition also comprises soybean oil or almond oil.

32. The method of claim 30 where the analgesic composition is applied to the selected area of skin one to four times per day.
